(19) United States
(12) Patent Application Publication
(10) Pub. No.: US 2003/0211045 A1
(43) Pub. Date: Nov. 13, 2003

(54) Magnetoliposome composition for targeted treatment of biological tissue and associated methods

(76) Inventors: Danuta Leszcyznska, Tallahassee, FL (US); Jerzy Leszczynski, Tallahassee, FL (US); Melania Babincova, Bratislava (SK); Peter Babinec, Bratislava (SK)

Correspondence Address: Enrique G Estevez, Allen Dyer Doppelt Milbrath & Gilchrist, 255 South Orange Avenue Suite 1401, PO Box 3791, Orlando, FL, 32802-3791, US

(21) Appl. No.: 10/182,992

(22) PCT Filed: Feb. 5, 2001
(86) PCT No.: PCT/US01/03738

(51) Int. Cl. A61K 49/00; A61K 38/38; A61K 38/17; A61K 31/716; A61K 31/715; A61K 39/395; A61K 31/4162; A61K 9/127

(52) U.S. Cl. 424/9.321; 424/450; 514/54; 514/2; 514/59; 514/60; 424/130.1; 514/8; 514/386

(57) ABSTRACT

A composition for treatment of biological tissue responsive to a an applied magnetic field comprises a plurality of magnetoliposomes, each magnetoliposome of the plurality having a lipid-containing wall defining a vesicle, and a plurality of subdomain superparamagnetic particles, and an inactive prodrug capable of activating into a drug effective for treatment of the biological tissue, the prodrug carried by the plurality of magnetoliposomes for delivery to the biological tissue. A method of treatment for a biological tissue comprises administering the composition to the tissue, concentrating the plurality of magnetoliposomes in the biological tissue responsive to a substantially constant magnetic field, activating the inactive prodrug into an effective drug by applying an electromagnetic field to the concentrated plurality of magnetoliposomes so as to therein generate heat sufficient for activation without appreciable rupture of individual magnetoliposomes of the plurality of magnetoliposomes, and releasing the activated effective drug into the biological tissue by sufficiently increasing permeability of the lipid-containing walls of individual magnetoliposomes of the plurality of magnetoliposomes to thereby release activated drug.

### RELATED APPLICATION

This application claims priority from co-pending provisional application Serial No. 60/180,494 which was filed on Feb. 5, 2000 and which is incorporated herein by reference in its entirety.

### FIELD OF THE INVENTION

The present invention relates to the field of drug therapy and, more particularly, to a composition for treatment of biological tissue responsive to a an applied magnetic field.

### BACKGROUND OF THE INVENTION

Cancer, which comprises a large and diverse group of diseases, results from the uncontrolled proliferation of cells. Continued cell division leads to the formation of tumors that invade adjacent normal tissues and organs and interfere with their function. In some cases, cancer cells become dislodged from their primary site and metastasize (spread) to other anatomic sites. Cancers can arise in almost any location in the body In the U.S., there are approximately 1.3 million new cases of cancer per year and the incidence of cancer is increasing. Over seven million people in the U.S. today have been diagnosed with cancer, resulting in estimated annual direct and indirect medical costs of over $50 billion associated with the management of cancer.

Once the extent of disease has been determined, cancer therapy typically includes some combination of surgery, radiation therapy, and/or chemotherapy. Unfortunately, many tumors are not controlled with surgery because of their size, their location, or the presence of metastases. In these cases, radiation therapy or chemotherapy are frequently used. Radiation therapy and chemotherapy destroy both healthy and diseased cells and cause serious side effects because their cytotoxic effects are not adequately selective. In addition to searching for new treatment approaches, substantial research in cancer treatment has been directed toward improving the efficacy and reducing toxicity of existing therapies. Radiation therapy is administered to the anatomic site where the tumor is located (known as the treatment field), while adjacent normal tissues are shielded as best possible to reduce radiation toxicity. This therapy is usually given several times per week over a period of two to six weeks. Irradiation of tissues with X-rays or gamma rays generates free radicals and electrons (highly reactive and short-lived molecules and particles) that attack intracellular molecules such as DNA and lead to cell death. Treatment planning and definition of the treatment field is highly dependent on imaging procedures that are required to determine the location and size of the tumor and its relationship to adjacent normal tissues. Radiation sensitizers are chemical or pharmacological agents that increase the lethal effects of radiation when administered in conjunction with it. Ideally, a radiation sensitizer should be safe, be simple to administer to the patient, and potentiate the effect of radiation at the tumor site and not the adjacent normal tissue. While there currently are no radiation sensitizers approved by the Food and Drug Administration (FDA), certain chemotherapy agents are frequently used off-label to increase the effectiveness of radiation therapy. However, the use of chemotherapy agents as radiation sensitizers has been limited by lack of tumor localization and by the systemic toxicity of these agents.

In the U.S., over 350,000 patients per year receive cytotoxic chemotherapy for treatment of many types of cancer. The effectiveness of chemotherapy agents usually is limited by their serious or life threatening side effects. These side effects often include nausea and vomiting, suppression of white blood cell and platelet counts, renal toxicity, pulmonary toxicity, neurotoxicity, and cardiac toxicity. Chemotherapy drugs distribute throughout the body in normal tissues as well as in the tumor. The cytotoxic effects to normal tissues is dose-limiting for most of these drugs, resulting in a very narrow therapeutic margin. Many recent advances in medical oncology have resulted from the discovery of certain drugs, such as anti-emetics and blood cell growth factors, that ameliorate the side effects of chemotherapy agents and allow for use of higher doses of chemotherapy. In addition, chemosensitizers are drugs which potentiate the anti-tumor activity of cancer chemotherapy agents. Although certain chemosensitizers have been tested experimentally, no such agents are yet approved by the FDA.

One of the promising recent techniques for treating tumors is photodynamic chemotherapy, an emerging cancer treatment based on the combined effects of visible light and a photosensitizing drug (photosensitizer) that is activated by exposure to light of a specific wavelength. In this procedure, a photosensitizer that accumulates in tumors is injected intravascularly into the patient. The tumor site is then illuminated with visible light of a particular energy and wavelength that is absorbed by the photosensitizer. The activated photosensitizer creates excited state oxygen molecules in those cells in which the drug has localized. These molecules are highly reactive with cellular components and cause tumor cell death. Preferably, a photosensitizer accumulates selectively in tumors and is capable of activation at a wavelength of light of about 700−80 nm. This wavelength will to a degree penetrate tissue, blood, and darkly pigmented skin in order to treat larger or more deeply situated tumors. Other important features include safety, lack of skin phototoxicity, and simple administration of the agent to the patient. In January of 1996, the FDA granted first approval for a photosensitizing agent for treatment of obstructing cancers of the esophagus. To date, however, photodynamic therapy has been restricted to treatment of superficial or small lesions because existing photosensitizers have been unable to absorb light of a wavelength capable of penetrating uniformly and deeply through tissues, blood, and pigmented melanomas. Other limitations of photosensitizers include unfavorable biolocalization, prolonged retention in the body during which many photosensitizers circulate through the blood and skin for up to 6 weeks, skin phototoxicity due to prolonged retention in the skin allowing for activation of the drug by ambient light, leading to severe burns to normal skin, and poor solubility in water, which complicates intravenous administration of the drug.

References

| U.S. Patents | | | |
|---|---|---|---|
| 4.101.435 | July, 1978 | Hasegawa et a. | 252/062 |
| 4,106,488 | August, 1978 | Gordon | 424/9 |
| 4,202,323 | May, 1980 | Zweig et al. | 128/1.1 |
| ?4,269,826 | January, 1981 | Widder et al. | 424/484 |
| 4,269,826 | May, 1981 | Zimmermann et al. | 424/1.17 |
| 4,345,588 | August, 1982 | Widder et al. | 600/12 |
| 4.501.726 | July, 1983 | Schroder et al. | 424/1.1 |
| 4,652,257 | March, 1985 | Chang | 604/052 |
| 4,558,690 | December, 1985 | Joyce | 128/898 |
| 4,801,459 | January, 1989 | Liburdy | 128/804 |
| 4,869,247 | September, 1989 | Howard et al. | 600/12 |
| 4,983,159 | January, 1991 | Rand | 600/9 |
| 4,989,601 | Febuary, 1991 | Marchosky et al. | 607/113 |
| 5,125,888 | June, 1992 | Howard et al. | 600/12 |
| 5,170,801 | December, 1992 | Casper et al. | 128/769 |
| 5,190,761 | March, 1993 | Liburdy | 424/450 |
| 5,545,395 | August, 1996 | Tournier et al. | 424/932 |
| 5,612,019 | March, 1997 | Gordon et al. | 424/9 |
| 5.622.686 | April, 1997 | Gordon et al. | 424/9 |
| 5,658,234 | August, 1997 | Dunlavy | 600/9 |
| 5,707,335 | January, 1998 | Howard et al. | 600/12 |
| 5,720,976 | Febuary, 1998 | Kim et al. | 424/450 |

Other Publications

Babincová M. Microwave induced leakage of magnetoliposomes. Possible clinical implications, Biolectrochem. Bioenerg. 1993, 32: 187.

Babincová M. Correlation between microwave-induced lipid peroxidation and liposome leakage, Z. Naturforsch. 1994, 49c:139

Babincová M. Microwave-controlled drug release from magnetoliposomes, Pharmazie 1995, 50: 702

Babincová M. and Babinec P. Possibility of magnetic targeting of drugs using magnetoliposomes, Pharmazie 1995, 50: 828.

Babincová M. and Babinec P., Controlled drug delivery using degradable magnetic polymers, Pharmazie 1996, 51: 515.

Babincová M. and Babinec P. Controlled drug delivery using magnetoliposomes Cell. Mol. Biol. Letters 1997, 2: 1.

Babincová M. and Machova E. Magnetoliposomes may be useful for the elimination of HIV from infected individuals Z. Naturforsch. 1998, 53c: xxx.

Bacon et al. Ferrite particles: a new magnetic resonance imaging contrast. Lack of acute or chronic hepatotoxicity after intravenous administration, J. Lab. Clin. Med. 1987, 110: 164.

Borrelli et al. Hysteresis heating for the treatment of tumors, Phys. Med. Biol. 1984, 29: 487.

Brown W. F. J. Appl. Phys. 1959, 30: 130.

Chapman D. Physiochemical properties of phopholipids and lipid water systems, Liposome Technology 1984 1: 1.

Crowe et al., Preservation of freeze-dried liposomes by trehalose, Archives of Biochemistry and Biophysics 1985, 242:240-247.

Deamer et al. Permeability of lipid bilayers to water and ionic solutes Chem. Phys. Lipids 1986 40:167.

Ferrucci J. T. and Stark D. D. Iron oxide-enhanced MR imaging of the liver and spleen: review of the first 5 years, Am. J. Roentgenol. 1990, 155: 943.

Gabizon et al. Liposome formlations with prolonged circulation time in blood and enhanced uptake by tumors, Proc. Natl. Acad. Sci., 1988, 85:6949.

Gregoriadis G. and Florence A. T. Liposomes in drug delivery. Clinical, diagnostic and ophtalmic potential, Drugs 1993, 45: 15.

Hafeli et al. Magnetically directed poly(lactic acid) Y(90)-microspheres: Novel agents for targeted intracavitary radiotherapy, J. Biomed. Mat. Res. 1994, 28: 901.

Hope et al., Production of large unilamellar vesicles by a rapid extrusion procedure. Characterization of size distribution, trapped volume and ability to maintain a membrane potential, Biochimica et Biophysica Acta, 1985, 812: 55.

Hope et al. Generation of multilamellar and unilamellar phospholipid vesicles Chem. Phys. of Lipids 1986 40: 89.

Jordan et al. Inductive heating of ferrimagnetic particles and magnetic fluids: physical evaluation of their potential for hyperthermia, Int. J. Hyperthermia 1991, 9: 51.

Liburdy R. P. and Magin R. L. Microwave stimulated drug release from liposomes, Radiation Res. 1985, 103: 266.

Lubbe et al. Preclinical experiences with magnetic drug targeting: Tolerance and efficacy, Cancer Res. 1996, 56: 4694.

Jain et al. Introduction to Biological Membranes Ch. 9 192-231 J. Wiley and Sons, NY 1980.

Magin R. L. and Morse P. D. Rapid measurement of drug release from temperature sensitive liposomes by electron paramegnetic resonance and radioisotope techniques, Biochim. Biophys. Acta 1983, 760: 357.

Mann et al. Formation of iron oxides in unilamellar vesicles, Journal of Colloid and Interface Science 1988, 122: 326.

Marsh D., CRC Handbook of Lipid Bilayers (CRC Press, Boca Raton, Fla. 1990) pp. 139.

Mathiowitz et al., Photochemical rupture of microcapsules: A model system, Journal of Applied Polymer Science, 1981, 26:809

Mathiowitz et al., Polyanhydride microspheres as drug carriers, Journal of Applied Polymer Science, 1988, 35:755.

Maruyama et al. Enhanced delivery of doxorubicin tu tumor by long-circulating thermosensitive liposomes and local hyperthermia, Biochim. Biophys. Acta 1993, 1149: 209.

Nayar et al. Generation of large unilamellar vesicles from long-chain saturated phosphtidylcholines by extrusion techniques, Biochim. Biophys. Acta 1989, 986: 200.

Neél L.: Ann. Géophys. 1949, 5: 99.

Peasley K. W. Destruction of HIV-infected cells by ferrofluid particles manipulated by an external magnetic field: Mechanical disruption and selective introduction of cytotoxic or antiretroviral substances into target cells, Med. Hypotheses 1996, 46:5.

Santaella et al. Extended in vivo blood circulation time of fluorinated liposomes, FEBS 1993, 336: 481.

Sato et al., Recent aspects in the use of liposomes in biotechnology and medicine, Prog. Lipid Res. 1992, 4: 345.

Shliomis et al. Magnetic properties of ferrocolloids, J. Magnet. Magnet. Mat. 1990, 85: 40.

Sinkula et al., Rationale for design of biologically reversible drug derivatives: Prodrugs, J. Pharm. Sci. 1975, 64: 181.

Szoka and Papahadjopoulos, Procedure for preparation of liposomes with large internal aqueous space and high capture by reverse-phase evaporation, Proc. Natl. Acad. Sci. 1978, 75: 4194.

Tomita et al. Temperature sensitive release of adriamycin, and amphiphilic antitumor agent, from dipalmitoylphosphatidylcholine-cholesterol liposomes, Biochim. Biophys. Acta 1989, 978: 185.

Viroonchatopan et al. Preparation and characterization of dextran magnetite-incorporated thermosensitive liposomes: an on-line flow system for quantifying magnetic responsiveness, Pharm. Res. 1995, 12: 1176.

Weisleder et al. Superparamagnetic iron oxide: pharmacokinetics and toxicity. Am. J. Roentgenol. 1989, 152: 167.

Widder et al. Tumor remision in Yoshida sarcoma-bearing rats by selective targeting of magnetic albumin microspheres containing doxorubicin, Proc. Natl. Acad. Sci. 1981,.78: 579.

Yanase et al. Intracellular hyperthermia for cancer using magnetite cationic liposomes: Ex vivo study, Jpn. J. Cancer Res. 1997, 88: 630.

Yatvin et al. Design of liposomes for enhanced local release of drugs by hyperthermia, Science 1978, 202: 1290.

Yerushalmi A. Combined treatment of solid tumor by local hyperthermia and actinomycin D, Br. J. Cancer 1978 37: 827.

### SUMMARY OF THE INVENTION

With the foregoing in mind, the present invention advantageously provides a therapeutic composition and drug delivery method potentially serving to minimize toxic side effects, lower the required dosage amounts, and decrease costs for the patient.

The present invention provides therapeutic drug composition for site-specific delivery of therapeutics. The composition for treatment of biological tissue responsive to a an applied magnetic field comprises a plurality of magnetoliposomes, each magnetoliposome of the plurality having a lipid-containing wall defining a vesicle, and a plurality of subdomain superparamagnetic particles, and an inactive prodrug capable of activating into a drug effective for treatment of the biological tissue, the prodrug carried by the plurality of magnetoliposomes for delivery to the biological tissue. Preferably, the inactive prodrug comprises an inactivating chemical group which is cleaved therefrom by heat to thereby generate an active drug.

A method aspect of the invention includes making a composition comprising a plurality of magnetoliposomes having a lipid-containing wall, a magnetic component, and an inactive prodrug for treatment of a predetermined tissue, the method comprising. A predetermined amount of phosphatidylcholine is dissolved in a solvent solution. The the mixture of phosphatidylcholine and solvent solution is evaporated until forming a thin lipid film. The the thin lipid film is hydrated with a buffered aqueous solution of dextran-magnetite and an inactive prodrug. The hydrated thin lipid film is shaken to form a plurality of magnetoliposomes encapsulating dextra-magnetite and inactive prodrug. The plurality of formed magnetoliposomes is then separated from excess dextra-magnetite and inactive prodrug.

The invention also provides a method of treatment including administering to the tissue a composition comprising a plurality of magnetoliposomes, each magnetoliposome of the plurality having a lipid-containing wall defining a vesicle, a plurality of subdomain superparamagnetic particles, and an inactive prodrug capable of activating into a drug effective for treatment of the biological tissue. The plurality of magnetoliposomes is concentrated in the biological tissue responsive to a substantially constant magnetic field. Following concentration in the tissue, the inactive prodrug is activated into an effective drug by applying an electromagnetic field to the concentrated plurality of magnetoliposomes so as to therein generate heat sufficient for activation without appreciable rupture of individual magnetoliposomes of the plurality of magnetoliposomes. The activated effective drug is then released into the biological tissue by applying an electromagnetic field to the concentrated plurality of magnetoliposomes having activated drug so as to therein generate heat sufficient for increasing permeability of the lipid-containing walls of individual magnetoliposomes of the plurality of magnetoliposomes thereby releasing activated drug. Additionally, the concentrated magnetoliposomes are preferably monitored for presence in the biological tissue before releasing to verify drug delivery thereto.

### BRIEF DESCRIPTION OF THE DRAWINGS

Some of the features, advantages, and benefits of the present invention having been stated, others will become apparent as the description proceeds when taken in conjunction with the accompanying drawings in which:

FIG. 1 illustrates an experimental animal having an external magnetic field applied to the area of the kidney, according to an embodiment of the present invention;

FIG. 2 displays concentration of magnetoliposomes in rat kidney tissue responsive to an applied magnetic field;

FIG. 3 shows an apparatus for measuring magnetic induction heating of a magnetoliposome composition according to the present invention;

FIG. 4 displays results of temperature increase of a magnetoliposome composition responsive to an applied high-frequency magnetic field; and

FIG. 5 shows results as for FIG. 4, wherein the magnetoliposome compositions comprise differing concentrations of magnetite.

### DETAILED DESCRIPTION OF THE PREFERRED EMBODIMENT

The present invention will now be described more fully hereinafter with reference to the accompanying drawings, in which preferred embodiments of the invention are shown. This invention may, however, be embodied in many different forms and should not be construed as limited to the illustrated embodiments set forth herein. Rather, these illustrated embodiments are provided so that this disclosure will be thorough and complete, and will fully convey the scope of the invention to those skilled in the art.

The present invention provides a targeted therapeutic drug delivery system comprising a magnetoliposome-comprising therapeutic composition, preferably having an inactive prodrug contained therein. A magnetoliposome is defined as a structure having a relatively spherical shape with an internal aqueous volume, formed from lipids (mainly phosphatidylcholines) with magnetic fluid encapsulated in the interior, adsorbed on the inner and/or outer lipid bilayer surface or embedded in the lipid bilayer. Similarly the inactive therapeutic compound may be embedded within the outer wall of the magnetoliposome, encapsulated in the magnetoliposome and/or attached to the magnetoliposome, as desired. Magnetoliposomes are also referred to herein as MLs.

For the generation of heat needed to activate prodrugs and to release activated agents from magnetoliposomes we suggest to use localized hyperthermia (i.e. heating of certain regions of the human body) using magnetic induction heating of magnetic fluids, which are suspensions of ferromagnetic or ferrite particles of size much smaller than a magnetic domain (1-100 nm). A carrier liquid (coatings) prevents the particles from aggregation. These subdomain superparamagnetic particles produces substantially more heat per unit mass than the 1000 times larger multidomain ferrite particles of similar composition, especially at low amplitudes of alternating magnetic field. The mechanism of heating is based on Brown effect (rotation of the particle as a whole according to external magnetic field) and Neél effect (Bloch wall motion within the ferromagnetic crystal). The magnetic fluids are physiologically well tolerated (e.g. for dextran-magnetite there is essentially no measurable LD50), as had been shown extensively on ferromagnetic contrast agents in MRI. Alternating magnetic field which is needed to heat magnetic fluid has the advantage that for the human body is almost “transparent”. If suitable frequencies and field strength combinations are used, no interaction is observed between the human body and the field; hence tolerable low power absorption is obtained (penetrance depth of light used in photodynamic chemotherapy is only ˜1-3 mm). The frequency of magnetic field should be greater than that sufficient to cause any appreciable neuromuscular response, and less than that capable of causing any detrimal eddy current heating or dielectric heating of healthy tissue (ideally from the frequency range 100-1000 KHz).

The magnetoliposomes of the present invention may be used for targeted therapeutic delivery either in vivo or in vitro. Preferably, each individual magnetoliposome is capable of activating and releasing substantially all of the therapeutic compound upon the application of alternating magnetic field. In certain embodiments, the release of all of the therapeutic compound from all of the magnetoliposomes is immediate; in other embodiments, the release is gradual. The preferred rate of release will vary depending upon the type of therapeutic application.

Magnetoliposomes can be prepared from various lipids. The activation of encapsulated drug should be preferably carried out at a temperature below the gel to liquid crystalline phase transition temperature of the lipid employed. By “gel to liquid crystalline phase transition temperature Tc”, it is meant the temperature at which a lipid bilayer will convert from a gel state to a liquid crystalline state. If a (magneto)liposomes with a predetermined phase transition temperature are heated above this temperature they release their content. The transition temperature of the liposomes depends unpon its lipid composition. Liposomes may be prepared from a variety of lipids including the following:

a, dimyristoylphosphatidylcholine (DMPC) Tc=24° C.

b, dipalmitoylphosphatidylcholine (DPPC) Tc=42° C.

c, distearoylphosphatidylcholine (DSPC) Tc=54° C.

d, diarachidoylphosphatidylcholine (DAPC) Tc=66° C.,

which are commercially available e.g. in Sigma (St. Louis, Mo.). Especially suitable are polymerizable or fluorinated analogues of lipids, where the phase transition temperature may be set to the value optimal for release of activated drug. Alternatively magnetoliposomes can be formed from lipid/polymer mixtures (e.g. N-isopropylacryalamide/ocatadecylacrylate/acrylic acid copolymer) so as to vary permeability of their membrane at various temperatures.

In preferred embodiments, the magnetoliposomes of the invention are stable, stability being defined as substantial resistance to rupture from the time of formation until the application of electromagnetic field. Further, the magnetoliposomes of the invention are preferably sufficiently stable in the vasculature such that they withstand recirculation. The magnetoliposomes may be coated such that uptake by the reticuloendothelial system is minimized. Useful coatings include, for example, gangliosides, glucuronate, galacturonate, guluronate, polyethyleneglycol, polypropylene glycol, polyvinylpyrrolidone, polyvinylalcohol, dextran,. starch, phosphorylated and sulfonated mono, di, tri, oligo and polysaccharides and albumin. The magnetoliposomes may also be coated for purposes such as evading recognition by the immune system, using e.g. non-ionic surfactant to produce a protective three-dimensional shell which renders particles almost undetectable by the macropahges, and magnetoliposomes are kept in the blood circulation for a long time.

Provided that the circulation half-life of the magnetoliposomes is sufficiently long, the magnetoliposomes will generally pass through the target tissue as they pass through the body. By focusing the release inducing alternating magnetic field on the selected tissue to be treated, the therapeutic will be released locally in the target tissue. As a further aid to targeting, antibodies, carbohydrates, peptides, glycopeptides, glycolipids and lectins may also be incorporated into the surface of the magnetoliposomes.

Chemical substances known as a prodrugs are well known in the art and include inactive drug precursors which, when exposed to high temperature, metabolizing enzymes, cavitation and/or pressure, in the presence of oxygen or otherwise, or when released from the magnetoliposomes, will form active drugs. Such prodrugs can be activated in the method of the invention, upon the application of alternating magnetic field to the prodrug-containing magnetoliposomes with the subsequent release from the magnetoliposomes. Suitable prodrugs will be apparent to those skilled in the art, and are described, for example, in Sinkula et al., J. Pharm. Sci. 1975 64, 181-210, the disclosure of which are hereby incorporated herein by reference in its entirety.

Prodrugs, for example, may comprise inactive forms of the active drugs wherein a chemical group is present on the prodrug which renders it inactive and/or confers solubility or some other property to the drug. In this form, the prodrugs are generally inactive, but once the chemical group has been cleaved from the prodrug by heat. Such prodrugs are well described in the art, and comprise a wide variety of drugs bound to chemical groups through bonds such as esters to short, medium or long chain aliphatic carbonates, hemi-esters of organic phosphate, pyrophosphate, sulfate, amides, amino acids, azo bonds, carbamate, phosphamide, glucosiduronate and N-acetylglucosamine.

Examples of prodrugs having a parent molecule reversibly linked to an inactivating chemical group are as follows: convallatoxin with ketals, hydantoin with alkyl esters, chlorphenesin with glycine or alanine esters, acetaminophen with caffeine complex, acetylsalicylic acid with THAM salt, acetylsalicylic acid with acetamidophenyl ester, naloxone with sulfate ester, procaine with polyethylene glycol, erythromycin with alkyl esters, clindamycin with alkyl esters or phosphate esters, tetracycline with betaine salts, 7-acylaminocephalosporins with ring-substituted acyloxybenzyl esters, nandrolone with phenylproprionate decanoate esters, estradiol with enol ether acetal, methylprednisolone with acetate esters, testosterone with n-acetylglucosaminide glucosiduronate (trimethylsilyl) ether, cortisol or prednisolone or dexamethasone with 21-phosphate esters. In addition, compounds which are generally thermally labile may be utilized to create toxic free radical compounds. Compounds with azolinkages, peroxides and disulfide linkages which decompose with high temperature are preferred. With this form of prodrug, azo, peroxide or disulfide bond containing compounds are activated by increased-heating produced via Brown and Neél effects to create cascades of free radicals from these prodrugs entrapped therein. A wide variety of drugs or chemicals may constitute these prodrugs, such as azo compounds, the general structure of such compounds being R—N═N—R, wherein R is a hydrocarbon chain, where the double bond between the two nitrogen atoms may react to create free radical products in vivo.

Exemplary drugs or compounds which may be used to create free radical products include azo containing compounds such as azobenzene, 2,2′-azobisisobutyronitrile, azodicarbonamide, azolitmin, azomycin, azosemide, azosulfamide, azoxybenzene, aztreonam, sudan III, sulfachrysoidine, sulfamidochrysoidine and sulfasalazine, compounds containing disulfide bonds such as sulbentine, thiamine disulfide, thiolutin, thiram, compounds containing peroxides such as hydrogen peroxide and benzoylperoxide, 2,2′-azobisisobutyronitrile, 2,2′-azobis(2-amidopropane) dihydrochloride, and 2,2′-azobis(2,4-dimethylvaleronitrile). Additionally, radiosensitizers such as metronidazole and misonidazole may be incorporated into the magnetoliposomes to create free radicals on thermal stimulation.

By way of an example of the use of prodrugs, an acylated chemical group may be bound to a drug via an ester linkage which would readily cleave in vivo by enzymatic action in serum. The acylated prodrug may be incorporated into the magnetoliposome. When the magnetoliposome bilayer is destroyed due to the electromagnetic heating, the prodrug encapsulated by the magnetoliposome will then be exposed to the serum. The ester linkage is then cleaved by esterases in the serum, thereby generating the drug.

The route of administration of the magnetoliposomes will vary depending on the intended use. Administration of therapeutic delivery systems of the present invention may be carried out in various fashions, such as intravascularly, intralymphatically, parenterally, subcutaneously, intramuscularly, intranasally, intrarectally, intraperitoneally, interstitially, into the airways via nebulizer, hyperbarically, orally, topically, or intratumorly, using a variety of dosage forms. One preferred route of administration is intravascularly. For intravascular use, the therapeutic delivery system is generally injected intravenously, but may be injected intraarterially as well. The magnetoliposomes of the invention may also be injected interstitially or into any body cavity.

Another aspect of the invention which enhances the effect of the therapeutic drug is mechanical obstruction of tumor tissue due to the concentration of magnetoliposomes in the tumor-feeding vasculature and the succesive necrosis of tumor body. Both hyperthermia and embolization synergically enhance the chemotherapeutic effect of activated drug. Moreover magnetoliposomes obstructing feeding vessels represents barrier which delay outflow of activated drug and minimize further adverse effects of a drug on healthy tissues.

Preparation of Magnetoliposomes:

100 mg of soy-bean phosphatidylcholine (Sigma) was dissolved in chloroform/methanol (2:1) in a round bottom flask and solvent was evaporated in a rotary evaporator, so that a thin lipid film was formed. This film was hydrated by 10 ml of Tris-saline buffer of pH 7.4 with a desired concentration of dextran-magnetite and flask was vigorously shaken. This procedure resulted in the formation of magnetoliposomes. Non-encapsulated magnetite particles were removed by magnetic decantation and centrifugation.

More specifically, magnetoliposomes were usually prepared by encapsulation of dextran stabilized magnetic fluid into the liposomes. The stabilized magnetite-dextran particles were preprared according to the following method.

(1) 3.8 grams of FeCl₃.6H₂O (3.8 g), 1.4 grams of FeCl₂.4H₂O (1.4 g) and 10 grams of dextran were dissolved in 75 milliliters (ml) of water with stirring via a magnetic stirring bar.

(2) 80 ml of 1 normal (1N) sodium hydroxide (NaOH) was added over a 30 minute time period, with vigorous-stirring. As the base was added, the solution changed in color from black to brown and then green. The final pH value of the solution was approximately 11.5.

(3) The solution from step (ii) was then heated with stirring to 80.degree. C. at which point the solution was dark brown. At this point, the pH of the solution was rapidly lowered to a value of 7 using 5N hydrochloric acid (HCl) with rapid cooling to below 10.degree. C. There was an appreciable amount of black magnetite on the magnetic stirring bar.

(4) The suspension from step (3) was then centrifuged for 1 hour at 5.degree. C. and 13,500 rpm in a DuPont Sorvall RC-5B refrigerated superspeed centrifuge. An appreciable amount of black magnetite was collected at the bottom of the centrifuge tube.

(5) The supernatant liquid collected from the tubes was passed through a 0.2 micron Nalgene filter. The filter was gray, indicating that the centrifuge allowed a small fraction having a particle diameter greater than 0.2 micron to remain in suspension.

(6) A 20 ml sample of the above was fractionated in a column (5 cm diameter, 40 cm length) packed with Sepharose, CL4B (Pharmacia), using a Tris buffer (3.075 g Tris base, 6.25 g NaCl, 24 ml of 1N HCl diluted to 1 liter) as the eluant. The flow rate was 4 ml per minute and a brown fraction was left at the upper part of the column. The total volume obtained in the fractionation collector was 90 ml.

(7) The middle 50 ml of the product from step (6) was dialyzed in the same Tris buffer using a dialysis membrane with a molecular weigh cutoff of 6000-8000 daltons. Dialysis was carried out at 5.degree. C.

(8) After 24 hours, the material was removed from the dialysis bag and concentrated in an Amicon diafiltration cell using a YM10 Diaflo filter (molecular weight cutoff of 10,000 daltons) and 25 pounds per square inch of nitrogen gas. The purified suspension was stored at 5° C. and was ready for applications.

Magnetic Field Generation:

For experiments we have used alternating magnetic fields with the amplitude 0-10 kA/m and frequencies 100 kHz-3.5 Mhz. Such fields were achieved inside the water-cooled cooper induction coil with radius r=10-20 cm (n=10-25 turns with turn to turn distance z=0.5-1 cm). The final geometry of the coil is selected to match the requirements for frequency and field strength. Inside the coil, a cylindrical Faraday shield is mounted to reduce the electric vector of the field and therefore the non-specific heating by dielectric losses. The coil constitutes part of a resonant tank with parallel matching to couple the tank to 50 Ohm output impedance of the high frequency power amplifier. Field strength inside the coil is calculated from coil geometry and coil voltage measurements. The inductor coil and other elements of a resonant tank are placed inside a grounded cage to reduce the field outside the working area to the levels acceptable by occupational safety and FCC regulations. The inductor coil was cooled with water and isolated from the MLs suspension by a foam covered tube. Temperature in the MLs suspension was measured using a nonabsorbing Vitek thermistor.

In recent years, the use of magnetic gradient fields for separation has become widespread in the fields of biology, biotechnology and other related disciplines. Applications include cell sorting, RNA and DNA isolation, preparation, purification and sequencing, as well as immunology and a wide variety of isolation techniques for biological entities. The two key magnetic components of such systems are the magnetic particles used in the separation of the biological entities, and the magnetic field used to separate them. Such a field is usually generated by permanents magnets, and sometimes electromagnets. Simple magnetic blocks typically generate field's gradients in the orders 1-6 T/m.

In the magnetic drug targeting is possible to used large variety of permanent magnets. We have obtained the best results using the SmCo magnets. Comparison of key characteristics of commercially available permanent magnets may be found in the Table 1.

TABLE 1

| Characteristic | Ceramic | Alnico | SmCo | NdFeB |
|---|---|---|---|---|
| Highest Energy | 32 | 59 | 254 | 382 |
| Product [kJm⁻³] | | | | |
| Maximum | 300 | 550 | 300 | 150 |
| Operating | | | | |
| Temperature | | | | |
| Resistance to | Moderate | Low | Very | High |
| Demagnetization | | | high | |
| Corrosion | Excellent | Excellent | Good | Poor |
| Resistance | | | | |
| Mechanical | Moderate | Tough | Very | Brittle |
| Toughness | | | brittle | |
| Relative cost | Very low | Moderate | Very | High |
| | | | high | |

### EXAMPLE 1

Concentration of MLs at a Target Organ

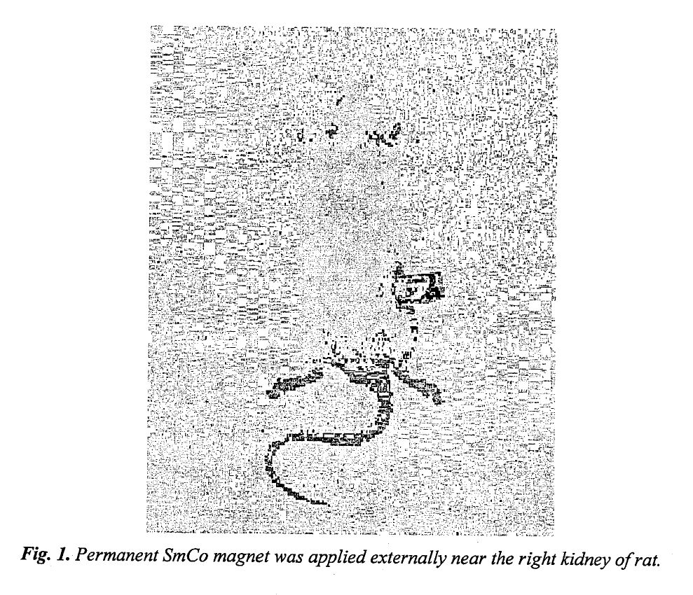

In a preliminary study, the kidney was chosen as a target organ from the point of view to open an avenue to treat venal tumors, which represents seventh leading cause of cancer. Some renal tumors e.g. rhabdoid kidney tumor are extremely aggressive and their therapy remains inadequate. Therefore delivering and confining chemotherapeutic agent to kidney could be effective for treatment of these malignancies. FIG. 1 illustrates the application of an external magnetic field to an experimental animal in this example.

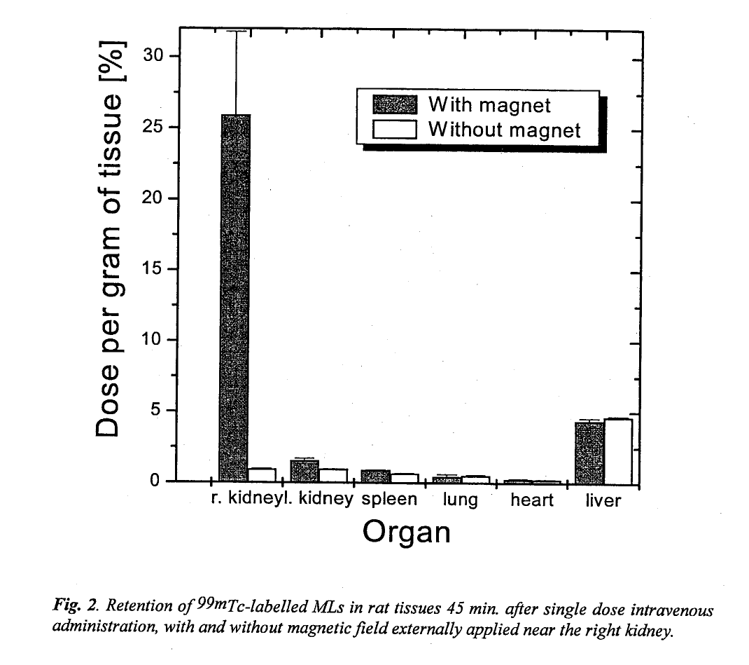

Shown in FIG. 2 is the distribution of the magnetoliposome composition in various tissues of experimental animals in this example. From the approximate expression for the force F_{mag} acting on ML

F_{mag}=V_{magnetite X}H(∂H/∂x)

where V_{magnetite} is the total volume of magnetite encapsulated in ML, _{X} is the magnetic susceptibility, H is the strength of magnetic field and (∂H/∂x) is magnetic field gradient is clear that the magnetic responsiveness of MLs is determined by many factors, including, strength of the applied magnetic field and the physical properties of encapsulated magnetic nanoparticles.

### EXAMPLE 2

Magnetic Induction Heating of MLs

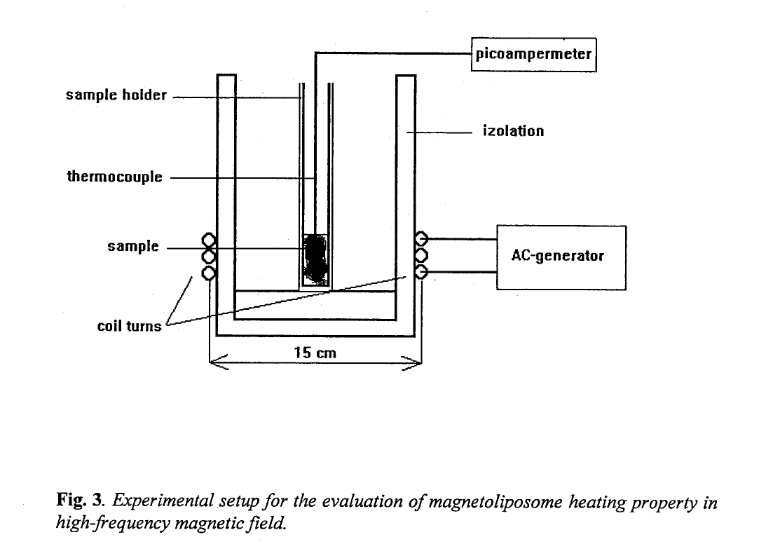

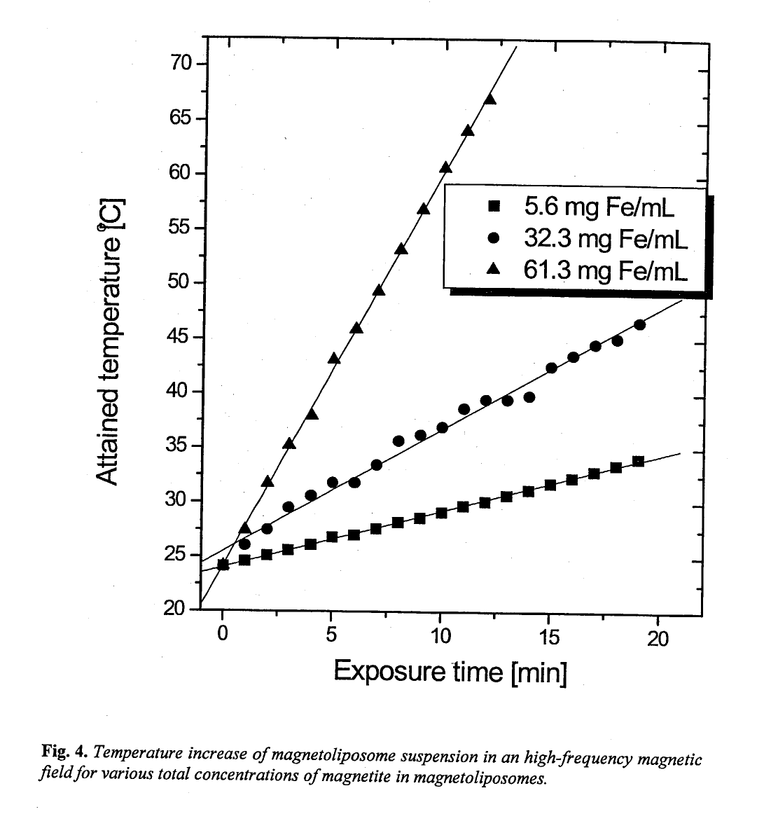

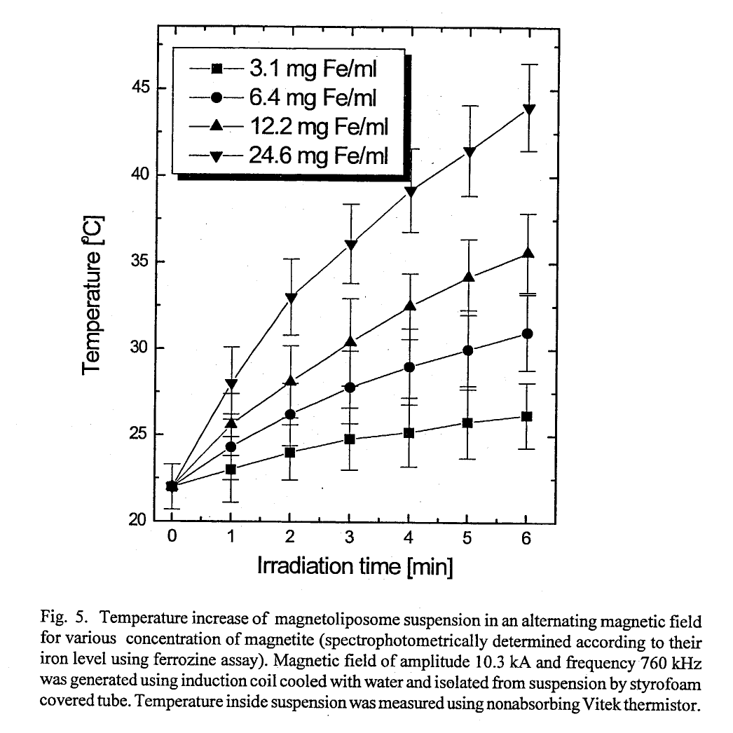

In further studies it has been shown that MLs encapsulated drug may be released in response to localized hyperthermia (i.e. heating of certain regions of the human body) using magnetic induction heating of magnetic fluids, which are suspensions of ferromagnetic or ferrite particles of size much smaller than a magnetic domain (1-100 nm). A carrier liquid (coatings) prevents the particles from aggregation. These subdomain superparamagnetic particles produces substantially more heat per unit mass than the 1000 times larger multidomain ferrite particles of similar composition, especially at low amplitudes of alternating magnetic field. The mechanism of heating is based on Brown effect (rotation of the particle as a whole according to external magnetic field) and Neél effect (Bloch wall motion within the ferromagnetic crystal). The magnetic fluids are physiologically well tolerated (e.g. for dextran-magnetite there is essentially no measurable LD50), as had been shown extensively on ferromagnetic contrast agents in MRI. Alternating magnetic field which is needed to heat magnetic fluid has the advantage that for the human body is almost “transparent”. If suitable frequencies and field strength combinations are used, no interaction is observed between the human body and the field; hence tolerable low power absorption is obtained (penetrance depth of light used in photodynamic chemotherapy is only ˜1-3 mm). The frequency of magnetic field should be greater than that sufficient to cause any appreciable neuromuscular response, and less than that capable of causing any detrimal eddy current heating or dielectric heating of healthy tissue. It has been found that a frequency range of about 100-1000 Khz is preferable. A system for evaluation of the thermal property of magnetoliposomes is shown in FIG. 3. The system consisted of a high-frequency producing unit (GV6A, ZEZ Rychnov, Czech Republic) with 6 kW generator, giving an alternating magnetic field with frequency 3.5 MHz and intensity 1.5 mT in three turn pancake coil, thermometry system consisting from copper-constantane thermocouple connected to voltmeter and reference stabilized thermal bath. Results of magnetic induction heating of magnetoliposomes is shown in FIGS. 4 and 5.

### EXAMPLE 3

Heating of MLs in Experimental Animals

In addition, we have performed also in vivo experiments to evaluate heating capabilities of MLs in living organisms. For these purposes BP-6 cells derived from a rat sarcoma induced by 3,4-benzpyrene were used. Adult female Sprague-Dawley rats 200 g in weight were inoculated with 2×10⁶ cells in 0.5 mL of saline subcutaneously in the right and left posterior flanks. Tumors were allowed to grow for 27 days when the average size in length and wide was 1.5 cm. Before the hyperthermic treatment the rats were anaesthetized and then 1 mL of MLs suspension in saline bufferwith total magnetite concentration 61.3 mg/mL was injected into the center of tumor using a 24-gauge needle. Rats with injected Mls were subsequently exposed to an alternating magnetic field. Temperature in the center of tumor was measured after the treatment by inserting thermocouple fiber into the desired site. Optimal increase of temperature to the 44.1° C. was achieved after 10 min exposure. We have measured also surface temperature in other parts of the body and also temperature in the center of tumor without the injected MLs using the same method, and as we have found that the initial rat temperature ≈35° C. increased at most by 2° C. These results therefore represent MLs as a promising material suitable for localized tumor treatment.

Another important factor which enhances the effect of chemotherapy and hyperthermia is mechanical obstruction of tumor due to the enhanced concentration of MLs in the tumor-feeding vessels and the succesive necrosis of tumor body. Both hyperthermia and embolization may synergically enhance the chemotherapeutic effect of released drug. Moreover MLs obstructing feeding vessels represents barrier which delay outflow of activated drug and minimize further adverse effects of a drug on healthy tissues.

Because the life-time of liposomes of this composition in the blood-stream is about one hour to study the influence of magnetic field on MLs in vivo distribution we have applied magnetic field generated by small permanent magnet fixed for 45 min near the right kidney. As shown in FIG. 2, the value of 25.92±5.84 % for magnetically targeted right kidney is significantly higher than 0.93±0.05% for non-targeted left kidney. The values for other studied organs are similar to that obtained in the MLs distribution study without magnetic field. The results of this study validate the usage of MLs for their targeting to desired sites in the body, on application of external magnetic field.

In the related studies we have shown that MLs encapsulated drug may be released in response to localized hyperthermia (i.e. heating of certain regions of the human body) using magnetic induction heating of magnetic fluids, which are suspensions of ferromagnetic or ferrite particles of size much smaller than a magnetic domain (1-100 nm). A carrier liquid (coatings) prevents the particles from aggregation. These subdomain superparamagnetic particles produces substantially more heat per unit mass than the 1000 times larger multidomain ferrite particles of similar composition, especially at low amplitudes of alternating magnetic field. The mechanism of heating is based on Brown effect (rotation of the particle as a whole according to external magnetic field) and Neél effect (Bloch wall motion within the ferromagnetic crystal). The magnetic fluids are physiologically well tolerated (e.g. for dextran-magnetite there is essentially no measurable LD50), as had been shown extensively on ferromagnetic contrast agents in MRI. The alternating magnetic field which is needed to heat the MLs has the advantage that for the human body is almost “transparent”. If suitable frequencies and field strength combinations are used, no interaction is observed between the human body and the field; hence tolerable low power absorption is obtained (penetrance depth of light used in photodynamic chemotherapy is only ˜1-3 mm). The frequency of magnetic field should be greater than that sufficient to cause any appreciable neuromuscular response, and less than that capable of causing any detrimal eddy current heating or dielectric heating of healthy tissue, preferably from about 100-1000 Khz.

In the drawings and specification, there have been disclosed a typical preferred embodiment of the invention, and although specific terms are employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

That which is claimed:

1. A composition for treatment of biological tissue responsive to a an applied magnetic field, said composition comprising:
a plurality of magnetoliposomes, each magnetoliposome of the plurality having a lipid-containing wall defining a vesicle, and a plurality of subdomain superparamagnetic particles; and
an inactive prodrug capable of activating into a drug effective for treatment of the biological tissue, said prodrug carried by said plurality of magnetoliposomes for delivery to the biological tissue.

2. The composition of claim 1, wherein said inactive prodrug comprises an inactivating chemical group which is cleaved therefrom by heat to thereby generate an active drug.

3. The composition of claim 1, comprising a chemical group rendering said prodrug inactive, said chemical group selected from an aliphatic carbon group, a phosphate group, a pyrophosphate group, a sulfate group, an amide group, an amino acid group, a carbamate group, a phosphamide group, a glucosiduronate group, and an N-acetylglucosamine group.

4. The composition of claim 1, wherein said inactive prodrug comprises an inactivating chemical group which is cleaved therefrom by an enzyme in the biological tissue to thereby generate an active drug.

5. The composition of claim 1, wherein said inactive prodrug comprises an acylated group bound by an ester linkage thereto.

6. The composition of claim 1, wherein said inactive prodrug comprises a plurality of prodrug components reactive with each other responsive to heat to thereby activate the inactive prodrug.

7. The composition of claim 1, comprising a magnetic fluid within the vesicle.

8. The composition of claim 1, wherein the lipid-containing wall comprises a phosphatidylcholine.

9. The composition of claim 1, wherein the lipid-containing wall comprises a lipid selected from dimyristoylphosphatidylcholine, dipalmitoylphosphatidylcholine, distearoylphosphatidylcholine, diarachidoylphosphatidylcholine, and a combination thereof.

10. The composition of claim 1, wherein the lipid-containing wall comprises a lipid analog.

11. The composition of claim 1, wherein the lipid-containing wall comprises a lipid analog selected from fluorinated lipid analogs and polymerizable lipid analogs.

12. The composition of claim 1, wherein the lipid-containing wall comprises a lipid and polymer mixture.

13. The composition of claim 1, wherein the lipid-containing wall is predetermined to impart each magnetoliposome of the plurality of magnetoliposomes with a desired permeability.

14. The composition of claim 1, wherein the lipid-containing wall comprises a lipid bilayer having an inner layer defining a periphery of the vesicle and an outer layer defining a periphery of the magnetoliposome.

15. The composition of claim 1, wherein the lipid-containing wall comprises a lipid bilayer having the plurality of subdomain superparamagnetic particles associated therewith.

16. The composition of claim 1, wherein the plurality of subdomain superparamagnetic particles comprises ferromagnetic particles.

17. The composition of claim 1, wherein the plurality of subdomain superparamagnetic particles comprises particles sized from about 1 to about 100 nanometers.

18. The composition of claim 1, further comprising a pharmaceutically acceptable carrier for reducing aggregation of the plurality of subdomain superparamagnetic particles.

19. The composition of claim 1, wherein the plurality of subdomain superparamagnetic particles comprises a dextran-magnetite fluid.

20. The composition of claim 1, further comprising a ferromagnetic contrast agent useful in magnetic resonance imaging.

21. The composition of claim 1, further comprising an agent substantially effective for reducing uptake of said plurality of magnetoliposomes by reticuloendothelial cells when the composition is administered to a patient.

22. The composition of claim 21, wherein said agent is selected from gangliosides, glucuronates, galacturonates, guluronates, polyethylene glycols, polypropylene glycols, polyvinypyrrolidones, polyvinyl alcohols, dextrans, starches, phosphorylated and sulfonated monosaccharides and polysaccharides, albumin, and combinations thereof.

23. The composition of claim 1, further comprising an agent substantially effective for reducing recognition of said plurality of magnetoliposomes by a patient's immune system when the composition is administered thereto.

24. The composition of claim 23, wherein said agent is selected from non-ionic surfactants and combinations thereof, and the composition is administered to the patient intravascularly.

25. The composition of claim 1, wherein the plurality of magnetoliposomes comprises a binding agent for the biological tissue.

26. The composition of claim 25, wherein said binding agent is associated with the lipid-containing wall of each individual magnetoliposome of the plurality of magnetoliposomes.

27. The composition of claim 25 wherein said binding agent is selected from antibodies, carbohydrates, peptides, polypeptides, glycopeptides, glycolipids, and lectins.

28. The composition of claim 1, further comprising a radiation sensitizer.

29. The composition of claim 28, wherein said radiation sensitizer is selected from metronidazole and misonidazole, or a combination thereof.

30. The composition of claim 1, further comprising a chemosensitizer potentiating the effect of a drug.

31. The composition of claim 30, wherein the chemosensitizer potentiates anti-tumor activity of a drug.

32. The composition of claim 1, comprising a pharmaceutically acceptable carrier for administration to a patient by a route selected from intravascularly, intralymphatically, parenterally, subcutaneously, intramuscularly, intranasally, intrarectally, intraperitoneally, intrathecally, interstitially, into the airways by nebulizer, hyperbarically, orally topically, intratumorly, by injection into a body cavity, and a combination thereof.

33. A method of treatment for a biological tissue, the method comprising:
administering to the tissue a composition comprising a plurality of magnetoliposomes, each magnetoliposome of the plurality having a lipid-containing wall defining a vesicle, a plurality of subdomain superparamagnetic particles, and an inactive prodrug capable of activating into a drug effective for treatment of the biological tissue;
concentrating the plurality of magnetoliposomes in the biological tissue responsive to a substantially constant magnetic field;
activating the inactive prodrug into an effective drug by applying an electromagnetic field to the concentrated plurality of magnetoliposomes so as to therein generate heat sufficient for activation without appreciable rupture of individual magnetoliposomes of the plurality of magnetoliposomes; and
releasing the activated effective drug into the biological tissue by sufficiently increasing permeability of the lipid-containing walls of individual magnetoliposomes of the plurality of magnetoliposomes to thereby release activated drug.

34. The method of claim 33, wherein releasing comprises applying an electromagnetic field to the concentrated plurality of magnetoliposomes having activated drug therein so as to generate heat sufficient for disrupting the lipid-containing wall.

35. The method of claim 33, wherein releasing comprises applying ultrasonic waves to the concentrated plurality of magnetoliposomes having activated drug therein so as to generate heat sufficient for disrupting the lipid-containing wall.

36. The method of claim 33, further comprising monitoring presence of the plurality of magnetoliposomes in the biological tissue before releasing to verify drug delivery thereto.

37. The method of claim 33, wherein concentrating comprises generating the constant magnetic field externally to the patient.

38. The method of claim 33, wherein concentrating comprises applying the constant magnetic field endoscopically to the patient.

39. The method of claim 33, wherein the inactive prodrug comprises a plurality of prodrug components and activating comprises a reaction between the plurality of prodrug components responsive to the heat generated.

40. The method of claim 33, wherein releasing comprises generating heat sufficient to increase permeability of individual magnetoliposomes of the plurality of magnetoliposomes without exceeding a gel to liquid crystalline phase transition temperature to thereby cause a relatively slow release of activated effective drug.

41. The method of claim 33, wherein releasing comprises generating sufficient heat to exceed a gel to liquid crystalline phase transition temperature for the plurality of magnetoliposomes to thereby cause a substantially immediate release of activated effective drug.

42. The method of claim 33, wherein activating and releasing comprise applying an electromagnetic field having a frequency greater than that causing appreciable neuromuscular response, and lower than that causing appreciable heating of substantially healthy tissue.

43. The method of claim 42, wherein the electromagnetic field comprises a frequency of from about 100 to about 1000 kHz.

44. The method of claim 33, wherein a human or animal comprises the biological tissue and administering comprises a route selected from intravascularly, intralymphatically, parenterally, subcutaneously, intramuscularly, intranasally, intrarectally, intraperitoneally, intrathecally, interstitially, into the airways by nebulizer, hyperbarically, orally topically, intratumorly, by injection into a body cavity, and a combination thereof.

45. The method of claim 33, wherein the biological tissue comprises vasculature, wherein administering comprises intravascular administration of the composition, and wherein concentrating comprises causing intravascular blockage in the biological tissue by the plurality of magnetoliposomes.

46. The method of claim 33, wherein releasing generates sufficient heat for hyperthermically enhancing treatment of the biological tissue by the activated drug.

47. A method of making a composition comprising a plurality of magnetoliposomes having a lipid-containing wall defining a vesicle, a plurality of subdomain superparamagnetic particles, and a prodrug capable of activating into a drug effective for treatment of a predetermined tissue, the method comprising:
dissolving a predetermined amount of phosphatidylcholine in a solvent solution;
evaporating the mixture of phosphatidylcholine and solvent solution until forming a thin lipid film;
hydrating the thin lipid film with a buffered aqueous solution of dextran-magnetite and an inactive prodrug;
shaking the hydrated thin lipid film to form a plurality of magnetoliposomes encapsulating dextran-magnetite and inactive prodrug; and
separating the plurality of magnetoliposomes from excess dextra-magnetite and inactive prodrug.

48. The method of claim 47, wherein the solvent solution comprises a solvent selected from chloroform, methanol, and mixtures thereof.

49. The method of claim 47, wherein the buffered aqueous solution comprises a tris-saline buffer.

50. The method of claim 47, wherein the buffered aqueous solution comprises a pH of about 7.4.

51. The method of claim 47, wherein separating comprises a procedure selected from magnetic decantation, centrifugation, and a combination thereof.
